(19) United States
(12) Patent Application Publication
(10) Pub. No.: US 2003/0018372 A1
(43) Pub. Date: Jan. 23, 2003

(54) Use of a light source for treatment of a skin disease and method for treatment of a skin disease

(76) Inventor: Johannes Junger, Geisenbrunn (DE)

Correspondence Address: Womble Carlyle Sandridge & Rice, PLLC, P.O. Box 7037, Atlanta, GA, 30357-0037, US

(21) Appl. No.: 10/187,325

(22) Filed: Jul. 1, 2002

(30) Foreign Application Priority Data
Jul. 6, 2001 (DE) 101 32 823.0

(51) Int. Cl. A61N 1/00

(52) U.S. Cl. 607/88

(57) ABSTRACT

The invention relates to a use of a light source for the treatment of a skin disease and a method for treatment of a skin disease by irradiation of a skin area with light of this light source, the light source comprising:

a gas-filled emission room at least partially transparent to light and having a region closed with a foil, and

an electron source disposed and formed such that electrons generated by the electron source enter the emission room through the foil and are able to cause light emission therein.

### CROSS-REFERENCE TO RELATED APPLICATIONS

This application claims convention priority from German patent application no. 101 32 823.0, with a filing date of Jul. 6, 2001. The specification and drawing of the European patent application are specifically incorporated herein by this reference.

### BACKGROUND OF THE INVENTION

The present invention relates to a use of a light source for the treatment of a skin disease and a method for treatment of a skin disease by irradiating a skin area with light of this light source.

It is known that certain skin diseases can be alleviated or even cured by means of irradiation with light of a certain wavelength or certain wavelength ranges. For example, in treating psoriasis, the irradiation of the affected skin areas with ultraviolet (UV) radiation in a wavelength range of 300 to 320 nm has proved. Up to now, for this purpose UV lamps or also lasers have been employed. However, the used UV lamps are disadvantageous in that they often have an undesired wideband light emission. Especially in light emission with light of a wavelength of about 250 nm, DNA damages can occur in the irradiation of the skin area, which can result in cancer disease. In addition, light of an undesired wavelength can also have other negative effects. Lasers are extremely expensive and unwieldy because of their size.

### SUMMARY OF THE INVENTION

Therefore, it is at least one object of the invention to provide a possibility by which a skin disease can be treated effectively, with low cost, without any damaging side effects and in a simple manner.

This object is achieved by the use of a light source for the treatment of the skin disease by irradiation of the skin area with light of this light source, wherein the light source comprises a gas-filled emission room at least partially transparent to light and having a region closed with a foil. The light source additionally comprises an electron source disposed and formed such that electrons generated by the electron source enter the emission room through the foil and are able to cause light emission therein.

Such a light source is described in the laid-open specification DE 44 38 407 for laboratory applications. Document DE 44 38 407 is expressly incorporated herein by reference, in its entirety. The light source has numerous properties making it particularly suitable for the treatment of a skin disease. It is small and therefore easy to manage, so that optionally it even can be carried by a patient and at any time used for the treatment of the skin disease. Additionally, the light source has a high efficiency and a price advantage over the light sources known for this use. Further, it can be appropriately adapted to the respective application field with respect to the desired wavelength or the desired wavelength range.

In a first embodiment, the light source can be formed such that UV light, especially light of approximately 308 nm and/or of approximately 193 nm, can be generated. For example, this can be achieved by filling the emission room with xenon and chlorine or with argon and fluorine, respectively. Especially light of approximately 308 nm is particularly suited to the treatment of psoriasis.

Furthermore, the light source can be formed such that light of a narrow spectral range can be generated. This is useful especially if with respect to a certain skin disease a certain spectral range has proven curing, while light of another spectral range has a negative effect.

The light emission can primarily be excimer radiation resulting from the deceleration of the electrons in dense gas. Excimers concern two-atom molecules existing only in the excited state. When these decay again, this is associated with light emission. As suitable gases, for example the noble gases are possible. Thus, the gas in the emission room can include helium and/or neon and/or argon and/or krypton and/or xenon. Besides, for example it can also include chlorine and/or fluorine. By the choice of the gases, the wavelength range of the light emission can be determined.

The excimer emission occurs in electron beam excitation of noble gases subject to a pressure of 0.2 bar and more. In an embodiment of the invention, a pressure of 0.5 to 30 bar, especially 2 bar, exists in the emission room. Such a relatively high gas pressure results in a high light yield since the formation rate of the excimer molecules increases quadratic with the pressure.

The electron source can include a thermionic emission cathode and/or a field emission cathode. Therein, the energy of the generated electrons can be 5 to 40 keV, especially 20 keV. Preferably, a pressure of 10-7 mbar can exist in the electron source. Today, such voltages and pressures for generating an electron beam are easily and securely controllable.

In an embodiment of the invention the foil is a foil manufactured based on silicon, wherein it can particularly be manufactured of silicon nitride, silicon carbide or polysilicon. Such a foil can be manufactured with micro-mechanical methods, for example by chemical vapor deposition (CVD) and selective etching or the like. Therein, the foil can have a thickness of 100 to 300 nm, especially 200 nm. Thereby, the energy loss of the penetrating electrons is extremely low. Further, supporting structures can be provided for supporting the foil.

According to the invention, the described light source can be employed particularly for the treatment of psoriasis. Besides, the light source can also be used for alleviating or curing in the treatment of neurodermatitis or also acne vulgaris. Apart from that, numerous other skin diseases are known to one skilled in the art, wherein the irradiation with light results in an improvement of the symptoms, as has been proven.

With respect to a concrete embodiment of the light source, reference is made to DE 44 38 407, in which an embodiment suitable for the invention with various alternative developments is described and presented in detail. For the treatment of the skin disease, the light source is positioned in a preset distance over a patient's skin area to be irradiated, such that this skin area can be irradiated with light of this light source for a preset time. The distance and the duration of the irradiation depend on the type of the skin disease and the symptoms.

We claim:

1. Use of a light source for the treatment of a skin disease by irradiation of a skin area with light of this light source, the light source comprising:
a gas-filled emission room at least partially transparent to light and having a region closed with a foil, and
an electron source disposed and formed such that electrons generated by the electron source enter the emission room through the foil and are able to cause light emission therein.

2. Use according to claim 1,
characterized in that
the light source is formed such that UV light, especially light of approximately 308 nm and/or of approximately 193 nm, can be generated.

3. Use according to claim 2,
characterized in that
the light source is formed such that light of a narrow spectral range can be generated.

4. Use according to claim 1,
characterized in that
the light emission is excimer radiation.

5. Use according to claim 1,
characterized in that
the gas in the emission room includes helium and/or neon and/or argon and/or krypton and/or xenon and/or chlorine and/or fluorine.

6. Use according to claim 1,
characterized in that
a pressure of 0.5 to 30 bar, especially 2 bar, exists in the emission room.

7. Use according to claim 1,
characterized in that
the electron source includes a thermionic emission cathode and/or a field emission cathode.

8. Use according to claim 1,
characterized in that
the foil is a foil manufactured based on silicon.

9. Use according to claim 1,
characterized in that
the foil has supporting structures.

10. Use according to claim 1,
characterized in that
the energy of the generated electrons is 5-40 keV, especially 20 keV.

11. Use according to claim 1,
characterized in that
the light source is formed such that light of a narrow spectral range can be generated.

12. Use according to any of the preceding claims for the treatment of psoriasis, neurodermatitis, or acne vulgaris.

13. Method for treatment of a skin disease by irradiating a skin area with light of a light source, the light source comprising a gas-filled emission room at least partially transparent to light and having a region closed with a foil, and an electron source disposed and formed such that electrons generated by the electron source enter the emission room through the foil and are able to cause light emission therein, wherein the light source is positioned in a preset distance over a patient's skin area to be irradiated, such that this skin area can be irradiated with light of this light source for a preset time.

14. Method according to claim 13 wherein the distance and the duration of the irradiation depend on the type of the skin disease and the symptoms.

15. Method according to claim 13 wherein the light source is formed such that UV light, especially light of approximately 308 nm and/or of approximately 193 nm, can be generated.

16. Method according to claim 14 wherein the light source is formed such that light of a narrow spectral range can be generated.

17. Method according to claim 13 wherein the light source is formed such that light of a narrow spectral range can be generated.

18. Method according to claim 13 wherein the light emission is excimer radiation.

19. Method according to claim 13 wherein the gas in the emission room includes helium and/or neon and/or argon and/or krypton and/or xenon and/or chlorine and/or fluorine.

20. Method according to claim 13 wherein a pressure of 0.5 to 30 bar, especially 2 bar, exists in the emission room.

21. Method according to claim 13 wherein the electron source includes a thermionic emission cathode and/or a field emission cathode.

22. Method according to claim 13 wherein the foil is a foil manufactured based on silicon.

23. Method according to claim 13 wherein the foil has supporting structures.

24. Method according to claim 13 wherein the energy of the generated electrons is 5-40 keV, especially 20 keV.

25. Method according to claim 13 wherein the method is used for the treatment of psoriasis.

26. Method according to claim 13 wherein the method is used for the treatment of neurodermatitis.

27. Method according to claim 13 wherein the method is used for the treatment of acne vulgaris.
